(19) United States
(12) Patent Application Publication
(10) Pub. No.: US 2025/0344908 A1
(43) Pub. Date: Nov. 13, 2025

(54) BATHTUB WITH AROMATHERAPY DEVICE

(71) Applicant: BAINS ULTRA INC., Lévis (CA)

(72) Inventors: Louis GENDREAU, Sainte-Marie (CA); Henry BRUNELLE, Québec (CA)

(21) Appl. No.: 18/869,345

(22) PCT Filed: Jul. 20, 2023
(86) PCT No.: PCT/CA2023/050975

§ 371 Date: Nov. 26, 2024

Related U.S. Application Data
(60) Provisional application No. 63/390,674, filed on Jul. 20, 2022.

(51) Int. Cl. A47K 3/00; A47K 3/02; A61L 9/12

(52) U.S. Cl. CPC A47K 3/001; A47K 3/02; A61L 9/12; A61L 2209/132; A61L 2209/134

(57) ABSTRACT

A bathtub includes a basin delimited by an upstanding peripheral wall and a bottom. A main drain is disposed in the bottom. An overflow drain has an overflow opening disposed in the upstanding peripheral wall and spaced apart from the bottom. An overflow cavity is adjacent the overflow opening. The overflow opening fluidly connects the overflow cavity to the basin. An overflow conduit fluidly connects the overflow opening to the main drain. A receptacle is at least partially disposed in the overflow cavity and configured to contain a fragrance solution. An emission device is coupled to the receptacle and electrically connected. A removable cover is configured to cover the overflow opening. The removable cover includes an aperture disposed above a rim of the receptacle.

### TECHNICAL FIELD

This disclosure generally relate to the field of bathtubs, and more particularly to bathtubs with aromatherapy devices.

### BACKGROUND

Aromatherapy devices disperse pleasing fragrances and provide various benefits. These devices are used, for instance, in households and at spas. A particular use for aromatherapy devices is to enhance a bathtub experience. However, the implementation of aromatherapy devices with bathtubs is often cumbersome and non-intuitive. Thus, improvements are needed.

### SUMMARY

In accordance with one aspect, there is provided a bathtub comprising: a basin delimited by an upstanding peripheral wall and a bottom; a main drain disposed in the bottom; an overflow drain having an overflow opening disposed in the upstanding peripheral wall and spaced apart from the bottom, an overflow cavity adjacent the overflow opening, the overflow opening fluidly connecting the overflow cavity to the basin, and an overflow conduit fluidly connecting the overflow opening to the main drain; a receptacle at least partially disposed in the overflow cavity and configured to contain a fragrance solution, an emission device coupled to the receptacle and electrically connected; and a removable cover configured to cover the overflow opening, the removable cover including an aperture disposed above a rim of the receptacle.

In an embodiment according to any of the previous embodiments, the emission device is electrically connected to an electrical wire extending in the overflow conduit.

In an embodiment according to any of the previous embodiments, the emission device is a vibration generator.

In an embodiment according to any of the previous embodiments, the receptacle is curved and protrudes outwardly from the overflow opening.

In an embodiment according to any of the previous embodiments, the overflow cavity incudes a seat configured for at least partially supporting the receptacle.

In an embodiment according to any of the previous embodiments, the removable cover is tethered to the receptacle.

In an embodiment according to any of the previous embodiments, the aperture in the removable cover is a horizontal slit-shaped aperture.

In an embodiment according to any of the previous embodiments, the emission device is removably coupled to the receptacle and removable from the overflow cavity.

In an embodiment according to any of the previous embodiments, the bathtub further comprises an electrical connector electrically connecting the emission device to an electrical power source.

In an embodiment according to any of the previous embodiments, the electrical connector is one of a magnetic connector, a pogo pin connector, and an inductive connector.

In an embodiment according to any of the previous embodiments, the bathtub further comprises an additional aperture in the removable cover, the additional aperture disposed below the aperture and fluidly connecting the basin to the overflow conduit.

In accordance with another aspect, there is provided a method for distributing fragrances from an overflow opening of an overflow drain of a bathtub, comprising: providing a receptacle at least partially disposed in an overflow cavity in the overflow drain, the overflow cavity fluidly coupled to the overflow opening; introducing a fragrance solution into the receptacle; providing an electric current to an emission device coupled to the receptacle; generating, via the emission device, fragrances from the fragrance solution in the receptacle; and distributing the fragrances through the overflow opening.

In an embodiment according to any of the previous embodiments, providing the electric current to the emission device includes connecting the emission device to an electrical wire extending in the overflow drain or to an electrical power source via one of a magnetic connector, a pogo pin connector, and an inductive connector.

In an embodiment according to any of the previous embodiments, the method further comprises covering the overflow opening with a removable cover, the removable cover having an aperture disposed above a rim of the receptacle.

In an embodiment according to any of the previous embodiments, distributing the fragrances through the overflow opening further includes distributing the fragrances through the aperture in the removable cover.

In an embodiment according to any of the previous embodiments, covering the overflow opening with the removable cover further includes tethering the removable cover to the receptacle via a tether.

In an embodiment according to any of the previous embodiments, providing the electric current to the emission device includes providing the electric current to a vibration generator.

In an embodiment according to any of the previous embodiments, providing the electric current to the vibration generator further includes providing the electric current to a piezoelectric generator.

In an embodiment according to any of the previous embodiments, providing the receptacle at least partially disposed in the overflow cavity further includes providing a curved receptacle protruding outwardly from the overflow opening.

In an embodiment according to any of the previous embodiments, providing the receptacle at least partially disposed in the overflow cavity further includes at least partially supporting the receptacle via a seat in the overflow cavity.

Many further features and combinations thereof concerning the present improvements will appear to those skilled in the art following a reading of the instant disclosure.

### BRIEF DESCRIPTION OF THE DRAWINGS

FIG. 1 is a perspective view of an example of a bathtub according to an embodiment of the present disclosure;

FIG. 2 is a front schematic view of the overflow drain of the bathtub of FIG. 1 with an aromatherapy device installed therein;

FIGS. 3A-3E are side schematic views of the aromatherapy device of FIG. 2 in different configurations;

FIG. 4 is a top schematic view of the aromatherapy device of FIG. 2;

FIG. 5 is a side schematic view of a bottom of an overflow drain of the bathtub of FIG. 1;

FIG. 6 is an enhanced perspective view of an example of a bathtub according to another embodiment of the present disclosure;

FIG. 7 is a partially exploded, perspective view of the overflow drain of the bathtub of FIG. 6 with an aromatherapy device installed therein; and

FIG. 8 is a partially exploded, rear perspective view of the aromatherapy device of FIG. 7.

### DETAILED DESCRIPTION

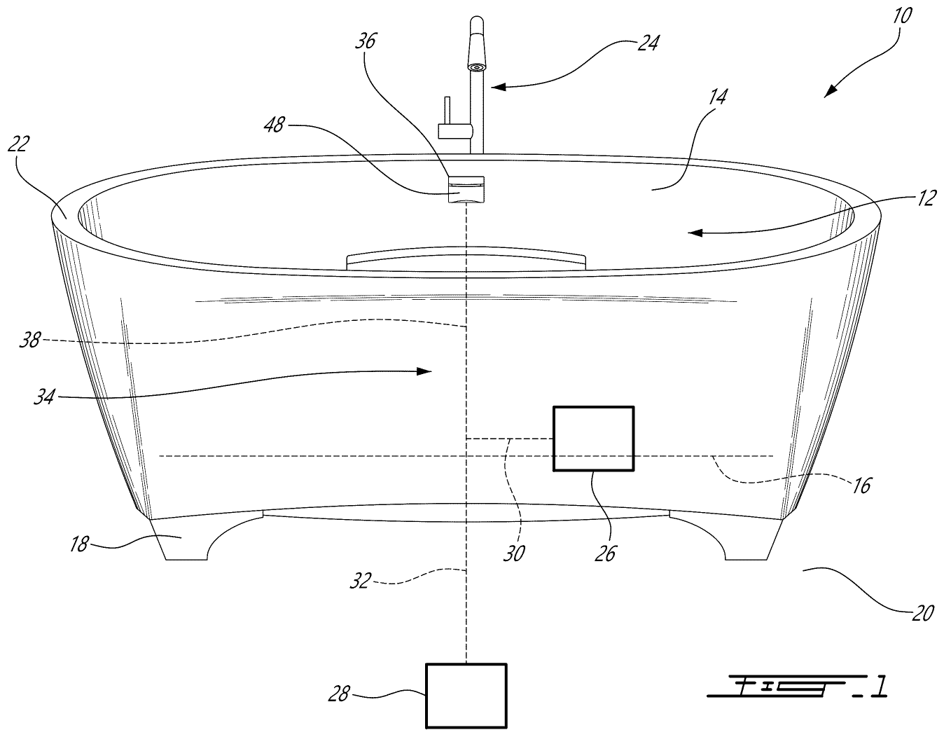

Referring to FIG. 1, there is shown a bathtub 10 in accordance with an embodiment of the present disclosure. In the shown case, the bathtub 10 is a freestanding bathtub, although other types of bathtubs may be contemplated, for instance alcove bathtubs, drop-in bathtubs, corner bathtubs, and combined shower-bathtubs, among others. Illustratively, the bathtub 10 includes a basin 12 delimited by an upstanding peripheral wall 14 and a bottom 16. A plurality of legs 18 illustratively support the bathtub above a floor 20, for instance a bathroom floor, or other like surface. An upper rim 22 of the bathtub 10 surrounds the upstanding peripheral wall 14 and defines an upper limit of the basin 12.

A faucet 24 provides a controlled quantity of water to the basin 12, while a main drain 26 disposed in the bottom 16 is configured for evacuating water, as well as other liquids, such as soap or shampoo, from the basin 12. The main drain 26 in fluidly connected to plumbing 28, for instance of a residence, hotel, spa, or other like location, via a main drain conduit 30 and plumbing conduit 32. The bathtub 10 further includes an overflow drain 34, i.e., a backup drain, for draining the basin 12 if the liquid in the basin 12 reaches a predetermined height in the basin 12 along the upstanding peripheral wall 14. The overflow drain 34 includes an overflow opening 36 (hidden in FIG. 1) disposed in the upstanding peripheral wall 14 and spaced apart from the bottom 16, preferably towards the upper rim 22. The overflow opening 36 is fluidly connected to the main drain 26 via an overflow conduit 38 and the main drain conduit 30.

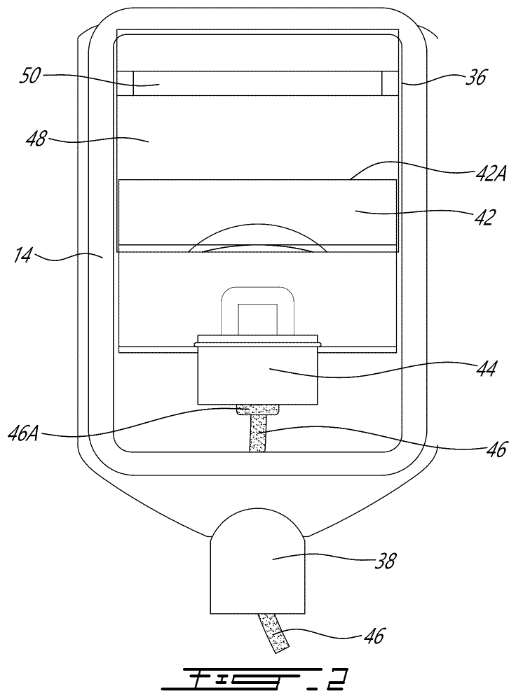

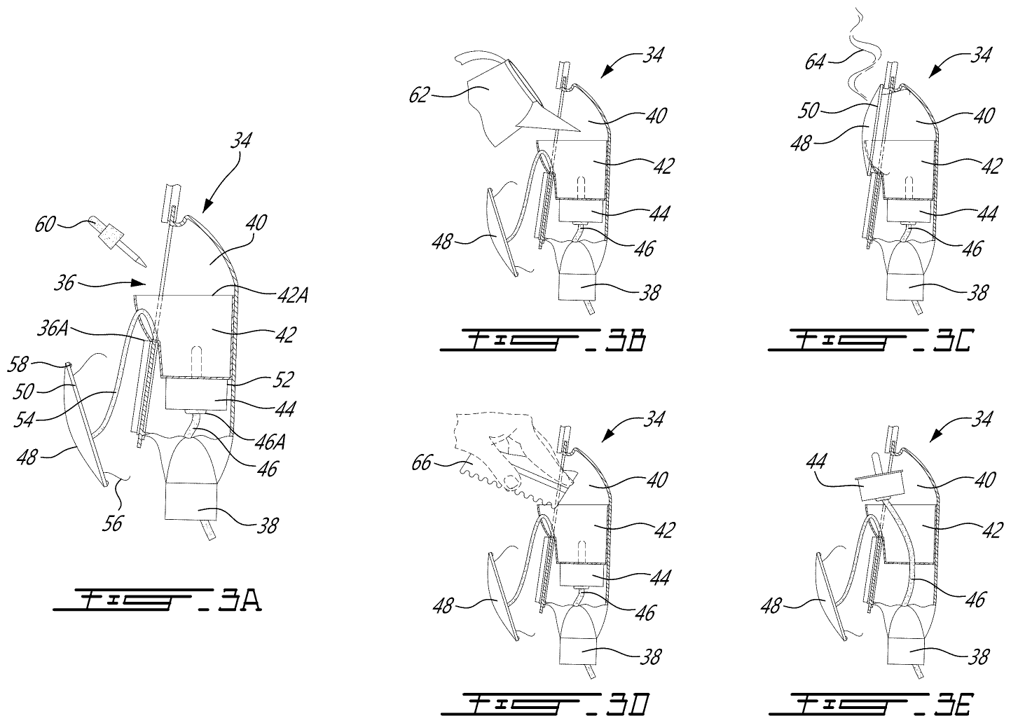

Referring to FIGS. 2 and 3A, the overflow drain 34 includes an overflow cavity 40 adjacent the overflow opening 36, illustratively at an upper end of the overflow conduit 38. A receptacle 42, with upper rim 42A and walls 42B, is at least partially disposed in the overflow cavity 40 and is configured to contain a fragrance solution, for instance a mixture of essential oils and water, although other fragrance solutions may be contemplated. An electrically-powered emission device 44 is coupled to the receptacle 42 and electrically connected to a source of electrical power (e,g, the power grid), for instance via an electrical wire 46 extending in the overflow conduit 38. Other means of electrical power provision may be contemplated. The emission device 44 is configured for generating fragrances from the fragrance solution in the receptacle 42 and emitting or distributing the fragrances through the overflow opening 36. As such, the bathtub 10 is configured for providing aromatherapy from a source of fragrance solution, i.e., the receptacle 42, disposed within the overflow drain 34. The bathtub 10 may thus be said to contain an integrated aromatherapy device, distributer, or diffuser.

Various types of emission devices 44 may be contemplated. In the shown case, the emission device 44 is a vibration generator, for instance a piezoelectric generator, configured for vibrating upon receiving an electric current via the electric wire 46. This vibration creates ultrasonic waves, thereby creating microscopic particles of the fragrance solution to be dispersed in the air through the overflow opening 36. In other cases, the emission device 44 may include a heater for heating and evaporating the fragrance solution so it may be dispersed. In other cases, wind, for instance generated by a fan, may be used to evaporate and disperse the fragrance solution. A nebulizer may additionally or alternatively be provided to atomize and disposer the fragrance solution. Various combinations of these types of emission devices 44, as well as other types of emission devices, may be contemplated.

A removable cover 48 may be provided to cover the overflow opening 36 and conceal the receptacle 42 within the overflow cavity 40. The removable cover 48 includes a body with an aperture 50 therethrough fluidly connecting the overflow cavity 40 to the basin 12. In the shown case, the aperture 50 is a horizontal, slit-shaped opening through the removable cover 48, although other aperture shapes may be contemplated. When the removable cover 48 is installed, fragrances generated by the emission device 44 are directed through the aperture 50. The receptacle 42, emission device 44 and removable cover 48 may thus be referred to as an aromatherapy device. In the shown case, the receptacle 42 has a curved shape, illustratively curved along two axes, protruding outwardly from the overflow cavity 40, with the removable cover 48 having a similarly curved or dome-like shape to surround the protruding portion of the receptacle 42. The receptacle 42 may be supported by a bottom edge or lip 36A of the overflow opening 36 and/or by a seat 52 in the overflow cavity 40. Other supporting means may be contemplated. The removable cover 48 may be tethered to the receptacle 42 via a tether 54, for instance a cord or cable. In some cases, the tether 54 may be removed from the removable cover 48 and/or the receptacle, for instance to replace the removable cover 48. In some cases, the tether 54 may be omitted. In the shown case, when the removable cover 48 is positioned to cover the overflow opening 36, the aperture 50 is disposed above the upper rim 42A of the receptacle 42.

Various connecting means may removably connect the removable cover 48 to the overflow opening 36. For instance, in the shown case, the removable cover 48 may include one or more (illustratively two) spring latches 56 that are latchable to the walls of the overflow cavity 40. In other cases, the removable cover 48 may include one or more magnets, with magnets of opposite polarity provided on the upstanding peripheral wall 14 adjacent the overflow opening 36 for securing the removable cover 48. Other joining means for the removable cover 48 may be contemplated. The removable cover 48 may be provided with sealing means, for instance an O-ring, to seal the removable cover 48 against the upstanding peripheral wall 14, thereby forcing the generated fragrance to exit the overflow cavity 40 through the aperture 50.

Referring additionally to FIGS. 3B-3E, various steps in preparing, using and servicing the fragrance emitting features of the bathtub 10 are shown. In FIGS. 3A and 3B, with the removable cover 48 removed from the overflow opening 36, a fragrance solution is introduced into the receptacle 42. Illustratively, a dropper 60 is shown in FIG. 3A for introducing essential oils into the receptacle 42, and a pitcher 62 is shown in FIG. 3B for introducing water into the receptacle 42, creating a mixture of water and essential oil in the receptacle 42. Other introduction means and/or types of fragrance-producing liquids may be contemplated. In FIG. 3C, the removable cover 48 is positioned to cover the overflow opening 36, with fragrances 64 (illustratively in evaporated form, generated by the emission device 44 in contact with the fragrance solution in the receptacle 42) exiting the overflow cavity 40 via the aperture 50 in the removable cover 48.

FIG. 3D shows an exemplary cleaning process for the receptacle 42, whereby a cleaning tool 66, illustratively a hand-held sponge, is used to clean the inside of the receptacle 42 while the removable cover 48 is removed. Other cleaning tools and processes may be contemplated. As shown in FIG. 3E, in some cases the emission device 44 may be removably coupled to the receptacle 42 and removable from the overflow cavity 40 via the overflow opening 36. For instance, removal of the emission device 44 may facilitate cleaning or servicing of the emission device 44. In other cases, the receptacle 42 and the emission device 44 may both be removable from the overflow cavity 40, either in a coupled or removably coupled fashion. The overflow opening 36 may thus be dimensioned so that the receptacle 42 and/or emission device 44 may pass therethrough. In some cases, the overflow opening 36 may be at least 2 inches high, with a width at least as great as its height. The removable cover 48 may be sized so that an inner side of its body occupies an area at least slightly greater than the area of the overflow opening 36 for adequate sealing thereof. In some embodiments, the electrical wire 46 is connected to the emission device 44 via a connector 46A, which may be configured to disconnect the emission device 44 from the electrical wire 46.

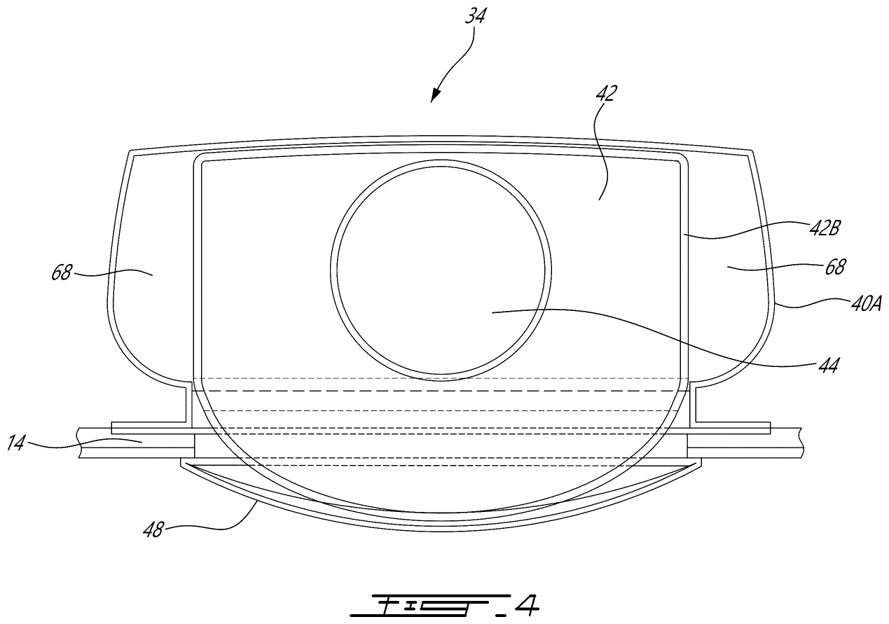

Referring to FIG. 4, a top view of the overflow drain 34 is shown. The overflow cavity 40 and receptacle 42 may be respectively sized so that a pair of lobes 68 are defined between the walls 42B of the receptacle 42 and inner walls 40A of the overflow cavity 40. The lobes 68 provide a conduit for liquid accumulated in the basin 12 to enter the overflow cavity 40, for instance via aperture 50, and flow through the overflow drain 34 towards the plumbing 28. In an exemplary embodiment, liquid accumulated in the basin 12 may enter the overflow cavity 40 via the aperture 50 in the removable cover 48, flow through the lobes 68 alongside the receptacle 42, and flow through the overflow conduit 38 towards the main drain 26 and plumbing 28. As the top end of the receptacle 42 may be uncovered or open-ended inside the overflow cavity 40, the flowing liquid from the basin 12 may additionally fill and overflow the receptacle 42 before flowing in the lobes 68 towards the main drain 26. the Other numbers of lobes 68 may be contemplated, and may depend on the shape of the receptacle.

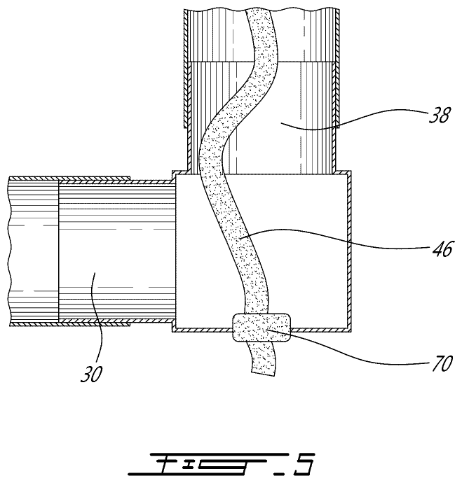

Referring to FIG. 5, an exemplary view of a bottom end of the overflow conduit 38 is shown. In the shown case, the overflow conduit 38 drains directly to the main drain 26 (not shown in FIG. 5) via main drain conduit 30, and the main drain 26 may drain directly to the plumbing 28 via plumbing conduit 32. In other cases, for instance as depicted in FIG. 1, the overflow conduit 38 may be directly fluidly connected to both the main drain 26 (via main drain conduit 30) and to the plumbing 28 (via plumbing conduit 32). In the shown case, the electrical wire 46 extending through the overflow conduit 38 exits the overflow conduit 38 at a lower end thereof via a plug or stopper 70. The stopper 70 may seal the overflow conduit 38, and may be removable so that the electrical wire 46 may be removed from the overflow conduit 38, for instance to be replaced. The stopper 70 may be adjacent a maintenance access door or access point in the upstanding peripheral wall 14 or underneath the bottom 16 of the bathtub 10, facilitating access to the electrical wire 46.

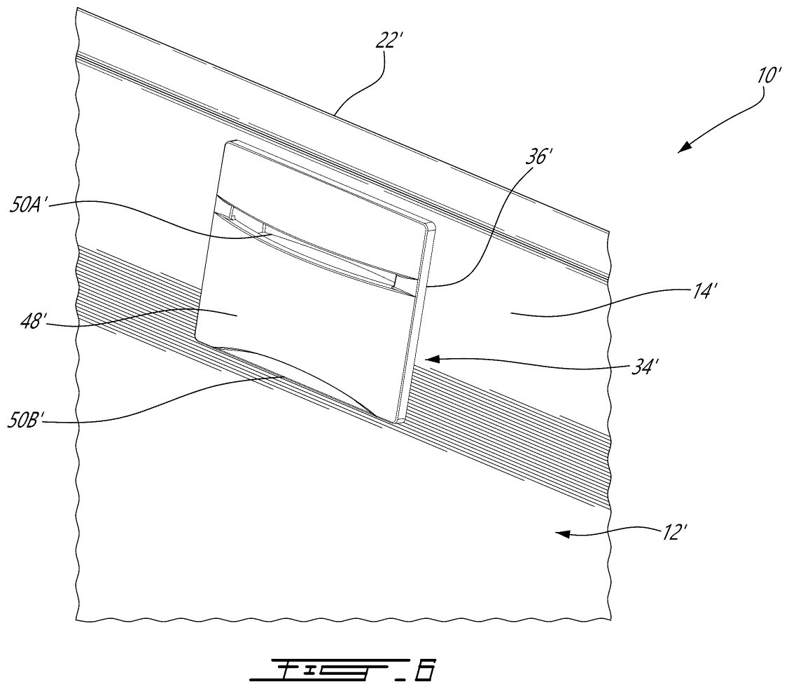

Referring to FIG. 6, there is shown a bathtub 10′ in accordance with another embodiment of the present disclosure, with like reference numerals referring to like elements. As in the embodiment of FIGS. 1-5, the bathtub 10′ is a freestanding bathtub, although other types of bathtubs may be contemplated, for instance alcove bathtubs, drop-in bathtubs, corner bathtubs, and combined shower-bathtubs, among others. Illustratively, the bathtub 10′ includes a basin 12′ delimited by an upstanding peripheral wall 14′ and a bottom (not shown). An upper rim 22′ of the bathtub 10′ surrounds the upstanding peripheral wall 14′ and defines an upper limit of the basin 12′. Various combinations of features from the bathtub 10 of FIGS. 1-5 and the bathtub 10′ of FIGS. 6-8 may be contemplated.

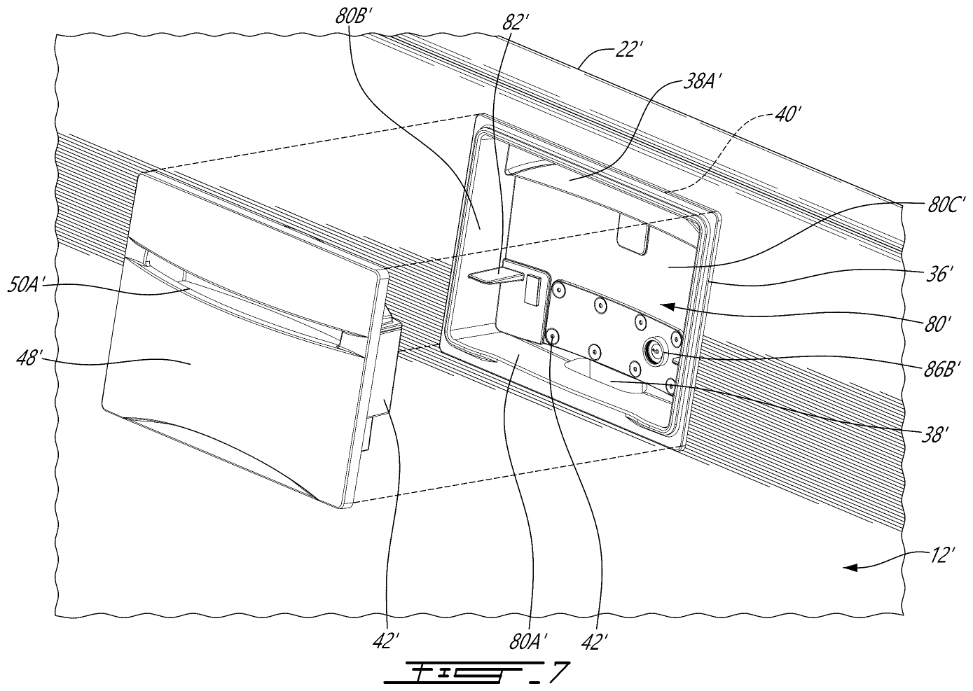

A faucet (not shown) may provide a controlled quantity of water to the basin 12′, while a main drain (not shown) disposed in the bottom is configured for evacuating water, as well as other liquids, such as soap or shampoo, from the basin 12′. The main drain in fluidly connected to plumbing, for instance of a residence, hotel, spa, or other like location, via a main drain conduit and plumbing conduit (not shown). The bathtub 10′ further includes an overflow drain 34′, i.e., a backup drain, for draining the basin 12′ if the liquid in the basin 12′ reaches a predetermined height in the basin 12′ along the upstanding peripheral wall 14′. The overflow drain 34′ includes an overflow opening 36′ disposed in the upstanding peripheral wall 14′ and spaced apart from the bottom, preferably towards the upper rim 22′. The overflow opening 36′ is fluidly connected to the main drain via an overflow conduit 38′ (see FIG. 7) and the main drain conduit.

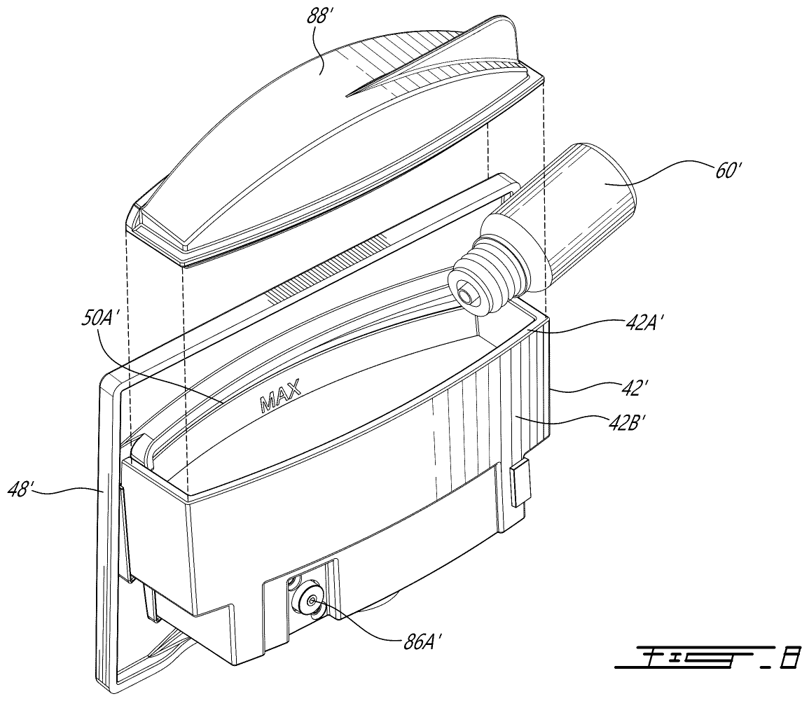

Referring to FIGS. 7 and 8, the overflow drain 34′ includes an overflow cavity 40′ adjacent the overflow opening 36′, illustratively at an upper end of the overflow conduit 38′. The insert 80′ is received within the overflow cavity 40′ and is configured for receiving a receptacle 42′, as will be discussed in further detail below. The insert 80′ may be have a rectangular or other cross-sectional shape and have bottom 80A′, top (not shown), side 80B′ and rear 80C′ walls. The overflow conduit 38′ may extend downwardly from the bottom wall 80A′, while an additional overflow conduit opening 38A′ disposed through an upper portion of the rear wall 80C′ may fluidly connect to the overflow conduit 38′. One or more fasteners 84′ may be provided for coupling the insert 80′ to the overflow drain 34′.

The receptacle 42′, with upper rim 42A′ and walls 42B′, is received by the insert 80′, for instance via tabs 82′ or other like retaining means, and is at least partially disposed in the overflow cavity 40′. The receptacle 42′ is configured to contain a fragrance solution, for instance a mixture of essential oils and water, although other fragrance solutions may be contemplated. An electrically-powered emission device (not shown) is coupled to the receptacle 42′ and electrically connected to a source of electrical power (e.g., the power grid). A first electrical connector 86A′ is illustratively disposed on the wall 42B′ of the receptacle 42′, while a second electrical connector 86B′ is illustratively disposed on the rear wall 80C′ of the insert 80′. Other positions for the electrical connectors 86A′, 86B′ may be contemplated. Various connection types between electrical connectors 86A′, 86B′ may be contemplated to provide electrical power to the emission device, for instance a magnetic connector, a pogo pin connector, or an inductive connector. Other connector types may be contemplated. An electrical wire (not shown) may extend from the second electrical connector 86B′ to the electrical power source. The electrical wire may, for instance, extend behind the upstanding peripheral wall 14′ of the bathtub 10′. Other paths for the electrical wire may be contemplated, for instance through the overflow conduit 38′. Power conversion means may be contemplated, for instance to reduce the voltage provided by the power source (e.g., at 120V) before it arrives at the emission device (e.g., at 24V). In other cases, the emission device may be powered by a battery disposable or storable inside or adjacent the overflow cavity 40′.

The emission device is configured for generating fragrances from the fragrance solution in the receptacle 42′ and emitting or distributing the fragrances through the overflow opening 36′. As such, the bathtub 10′ is configured for providing aromatherapy from a source of fragrance solution, i.e., the receptacle 42′, disposed within the overflow drain 34′. The bathtub 10′ may thus be said to contain an integrated aromatherapy device, distributer, or diffuser.

Various types of emission devices may be contemplated. For instance, the emission device may be a vibration generator, for instance a piezoelectric generator, configured for vibrating upon receiving an electric current via the electrical connectors 86A′, 86B′. This vibration creates ultrasonic waves, thereby creating microscopic particles of the fragrance solution to be dispersed in the air through the overflow opening 36′. In other cases, the emission device may include a heater for heating and evaporating the fragrance solution so it may be dispersed. In other cases, wind, for instance generated by a fan, may be used to evaporate and disperse the fragrance solution. A nebulizer may additionally or alternatively be provided to atomize and disposer the fragrance solution. Various combinations of these types of emission devices, as well as other types of emission devices, may be contemplated.

A removable cover 48′ may be provided to cover the overflow opening 36′ and conceal the receptacle 42′ within the overflow cavity 40′. The removable cover 48′ illustratively includes a body with first and second aperture 50A′, 50B′ therethrough to fluidly connect the overflow cavity 40′ to the basin 12′. In the shown case, the first aperture 50A′ is a horizontal, slit-shaped opening through the removable cover 48′, although other aperture shapes may be contemplated. When the removable cover 48′ is installed, fragrances generated by the emission device are directed through the first aperture 50A′. The receptacle 42′, emission device and removable cover 48′ may thus be referred to as an aromatherapy device. Various shapes for the receptacle 42′ and cover 48′, and various attachment means between the receptacle 42′ and cover 48′, may be contemplated. The receptacle 42′ may be supported by the bottom wall 80A′ of the insert 80′ and/or by the tabs 82′ or other like retaining means in the insert 80′. Other supporting means may be contemplated. In some cases, the removable cover 48′ may be tethered to the receptacle 42′, for instance by a cord or cable. In the shown case, when the removable cover 48′ is positioned to cover the overflow opening 36′, the first aperture 50A′ is disposed above the upper rim 42A′ of the receptacle 42′. The second aperture 50B′, which may be a gap or slit between the cover 48′ and the wall 14′, is illustratively disposed below the receptacle 42′ and above the overflow conduit 38′ when the removable cover 48′ is installed. As such, if water were to rise in the basin 12′ to the height of the overflow drain 34′, the water may first be diverted through the second aperture 50B′ towards the overflow conduit 38. The first aperture 50A′ may thus serve primarily as a fluid conduit for the emitted aromatherapy, and serve as a fluid conduit for rising water in the basin 12′ towards the overflow conduit 38′ via additional overflow conduit opening 38A′ if needed (i.e., if water were to rise higher than the height of the second aperture 50B′ in the basin 12′).

Referring to FIG. 8, various steps in preparing, using and servicing the fragrance emitting features of the bathtub 10′ are shown. With the removable cover 48′ and receptacle 42′ removed from the overflow opening 36′ and a top receptacle cover 88′ removed, a fragrance solution is introduced into the receptacle 42′. Illustratively, a dropper 60′ is shown in FIG. 8 for introducing essential oils into the receptacle 42′. A pitcher (not shown) may also be used to introduce water into the receptacle 42′, thereby creating a mixture of water and essential oil in the receptacle 42′. Other introduction means and/or types of fragrance-producing liquids may be contemplated. The receptacle 42′ may have a maximum capacity for fragrance-producing liquid that can be poured therein (denoted by the MAX line in FIG. 8). In the shown embodiment, the removable cover 48′ is positioned to cover the overflow opening 36′, with fragrances (for instance in evaporated form, generated by the emission device in contact with the fragrance solution in the receptacle 42′) exiting the overflow cavity 40′ via the first aperture 50A′ in the removable cover 48′. The receptacle 42′ may be cleaned in a similar manner to that shown in FIG. 3D and described above.

According to the present disclosure, there is shown an exemplary method for distributing fragrances 64 from an overflow opening 36, 36′ of an overflow drain 34, 34′ of a bathtub 10, 10′. A receptacle 42, 42′ is provided, the receptacle 42, 42′ at least partially disposed in an overflow cavity 40, 40′ in the overflow drain 34, 34′, the overflow cavity 40, 40′ fluidly coupled to the overflow opening 36, 36′. A fragrance solution is introduced into the receptacle, for instance via dropper 60, 60′ and/or pitcher 62. An electric current is provided, for instance via electric wire 46 and/or electrical connectors 86A′/86B′, to an emission device 44, for instance a vibration generator such as a piezoelectric generator, coupled to the receptacle 42, 42′. Fragrances 64 are generated, via the emission device 44, from the fragrance solution in the receptacle 42, 42′. The fragrances 64 are then distributed through the overflow opening 36, 36′, for instance through an aperture 50, 50A′ of a removable cover 48, 48′ covering the overflow opening 36, 36′. Various modifications and additions to the above-described method steps may be contemplated.

As can be seen therefore, the examples described above and illustrated are intended to be exemplary only. The scope is indicated by the appended claims.

1. A bathtub comprising:
a basin delimited by an upstanding peripheral wall and a bottom;
a main drain disposed in the bottom;
an overflow drain having an overflow opening disposed in the upstanding peripheral wall and spaced apart from the bottom, an overflow cavity adjacent the overflow opening, the overflow opening fluidly connecting the overflow cavity to the basin, and an overflow conduit fluidly connecting the overflow opening to the main drain;
a receptacle at least partially disposed in the overflow cavity and configured to contain a fragrance solution, an emission device coupled to the receptacle and electrically connected; and
a removable cover configured to cover the overflow opening, the removable cover comprising an aperture disposed above a rim of the receptacle.

2. The bathtub as defined in claim 1, wherein the emission device is electrically connected to an electrical wire extending in the overflow conduit.

3. The bathtub as defined in claim 1 or 2, wherein the emission device is a vibration generator.

4. The bathtub as defined in claim 1- or 2, wherein the receptacle is curved and protrudes outwardly from the overflow opening.

5. The bathtub as defined in claim 1- or 2, wherein the overflow cavity comprises a seat configured for at least partially supporting the receptacle.

6. The bathtub as defined in claim 1, wherein the removable cover is tethered to the receptacle.

7. The bathtub as defined in claim 1, wherein the aperture in the removable cover is a horizontal slit-shaped aperture.

8. The bathtub as defined in claim 1, wherein the emission device is removably coupled to the receptacle and removable from the overflow cavity.

9. The bathtub as defined in claim 2, further comprising an electrical connector electrically connecting the emission device and the electrical wire.

10. The bathtub as defined in claim 9, wherein the electrical connector is one of a magnetic connector, a pogo pin connector, or an inductive connector.

11. The bathtub as defined in claim 1, further comprising an additional aperture in the removable cover, the additional aperture disposed below the aperture and fluidly connecting the basin to the overflow conduit.

12. A method for distributing fragrances from an overflow opening of an overflow drain of a bathtub, comprising:
providing a receptacle at least partially disposed in an overflow cavity in the overflow drain, the overflow cavity fluidly coupled to the overflow opening;
introducing a fragrance solution into the receptacle;
providing an electric current to an emission device coupled to the receptacle;
generating, via the emission device, fragrances from the fragrance solution in the receptacle; and
distributing the fragrances through the overflow opening.

13. The method as defined in claim 12, wherein providing the electric current to the emission device comprises connecting the emission device to an electrical wire extending in the overflow drain or to an electrical power source via one of a magnetic connector, a pogo pin connector, or an inductive connector.

14. The method as defined in claim 12, further comprising covering the overflow opening with a removable cover, the removable cover having an aperture disposed above a rim of the receptacle.

15. The method as defined in claim 14, wherein distributing the fragrances through the overflow opening further comprises distributing the fragrances through the aperture in the removable cover.

16. The method as defined in claim 14, wherein covering the overflow opening with the removable cover further comprise tethering the removable cover to the receptacle via a tether.

17. The method as defined in claim 12, wherein providing the electric current to the emission device comprises providing the electric current to a vibration generator.

18. The method as defined in claim 17, wherein providing the electric current to the vibration generator further comprises providing the electric current to a piezoelectric generator.

19. The method as defined in claim 12, wherein providing the receptacle at least partially disposed in the overflow cavity further comprises providing a curved receptacle protruding outwardly from the overflow opening.

20. The method as defined in claim 12, wherein providing the receptacle at least partially disposed in the overflow cavity further comprises at least partially supporting the receptacle via a seat in the overflow cavity.
